SEQUENCE LISTING (18 sequences)

SEQ ID NO: 1 - DNA, 20 bp, Artificial
  primer
gtgaggatcaaaaactgggg

SEQ ID NO: 2 - DNA, 18 bp, Artificial
  primer
acctgcaggttggaccac

SEQ ID NO: 3 - DNA, 20 bp, artificial
  primer
cgtggtgacaagggtgagac

SEQ ID NO: 4 - DNA, 20 bp, artificial
  primer
taggtgatgttctgggaggc

SEQ ID NO: 5 - DNA, 24 bp, artificial
  primer
ctggctcccaacactgccaacgtc

SEQ ID NO: 6 - DNA, 24 bp, artificial
  primer
tcctttgggtttgcaacggattgt

SEQ ID NO: 7 - DNA, 20 bp, artificial
  primer
cactgttaccgccacttccc

SEQ ID NO: 8 - DNA, 20 bp, artificial
  primer
gagatcgttcctctcgccct

SEQ ID NO: 9 - DNA, 20 bp, artificial
  primer
agatccaggaaaggagggct

SEQ ID NO: 10 - DNA, 17 bp, artificial
  primer
aaagctctctgtagcct

SEQ ID NO: 11 - DNA, 24 bp, artificial
  primer
gatgcctggaatgccatccccgat

SEQ ID NO: 12 - DNA, 24 bp, artificial
  primer
aggcttggtgggattggaggggga

SEQ ID NO: 13 - DNA, 25 bp, artificial
  primer
gaggttcgacgtgaaggcgcagatg

SEQ ID NO: 14 - DNA, 25 bp, artificial
  primer
cataggtcacgtagcccacttggtc

SEQ ID NO: 15 - DNA, 19 bp, artificial
  primer
cagagacccacccccagca

SEQ ID NO: 16 - DNA, 20 bp, artificial
  primer
ctgtttgtgtttggcttgac

SEQ ID NO: 17 - DNA, 25 bp, artificial
  primer
caggtcatcacyatyggcaatgagc

SEQ ID NO: 18 - DNA, 25 bp, artificial
  primer
cggatgtcmacgtcacacttcatga

(19) United States
(12) Patent Application Publication
(10) Pub. No.: US 2006/0216363 A1
(43) Pub. Date: Sep. 28, 2006

(54) Novel herbal composition

(76) Inventors: Su Ying Liu, Taipei Shien (TW); Yi Ming Tsai, Taipei Shien (TW)

Correspondence Address: HARNESS, DICKEY & PIERCE, P.L.C., P.O. BOX 8910, RESTON, VA, 20195, US

(21) Appl. No.: 11/091,000

(22) Filed: Mar. 28, 2005

(51) Int. Cl. A61K 36/28; A61K 36/185

(52) U.S. Cl. 424/725; 424/764

(57) ABSTRACT

The present invention relates to herbal compositions comprising Lactuca sativa L., Aquilaria agallocha Roxb., and Atractylodes macrocephala Koidz., and methods of preventing or treating diseases or disorders caused by degeneration and/or inflammation.

### FIELD OF THE INVENTION

The present invention concerns a series of novel herbal combination compositions, methods of processing the compositions and methods of treating and preventing arthritis, repairing of articular joint surfaces and relief of symptoms associated with arthritis in humans and animals by the compositions of the invention.

### DESCRIPTION OF PRIOR ART

In 2001, the estimated prevalence rate of arthritis in the United States was 33%, that was 69.9 million adults (Bolen J et al. MMWR 2002; 51(42): 948-950). This included 10.6% of adults with arthritis, 10% with chronic joint symptoms, and 12.4% with both. From 2005 to 2030, the population of the US aged 65 or more is expected to increase from 12.9% to 20% and the prevalence rate of arthritis will be doubled to 41.1 million in 2030 (Hootman J M et al. MMWR 2003; 52(21): 489-491). The medical and societal costs of arthritis are enormous. In 1995, the medical care costed $22 billion, and total costs, including medical care and loss of productivity, exceeded $82 billion.

Arthritis is a disorder of the musculoskeletal system. The etiologies of arthritis are multifactorial, some well-defined, others still unclear. There are two common types of arthritis; inflammatory and non-inflammatory. The inflammatory type includes rheumatoid arthritis (RA), juvenile rheumatoid arthritis (JRA), psoriatic arthritis, Reiter's syndrome (reactive arthritis), Crohn's disease, ulcerative colitis and sarcoidosis, of which RA is the most commonly known.

RA is an autoimmune disease with the prevalent rate of about 1.9% in persons 60 years of age or older in the US (Rasch E K, et. al. 2003, Arthritis & Rheumatism 48(4): 917-926), occurring two to three times more often in women than in men. RA is a chronic disease and typically affects many joints. It is characterized by inflammation of the membrane lining joints (synovium), which causes pain, stiffness, warmth, redness and swelling. The inflamed synovium can invade and damage cartilage and bone.

The non-inflammatory arthritis, represented by osteoarthritis (OA), or wear and tear degenerative arthritis, is characterized by joint pain and limited movement resulted from progressive loss of articular cartilage. OA is the most common type of arthritis, especially among elderly. It was estimated that 12.1% of Americans aged 25 and older show clinical signs or symptoms of OA (Lawrence R C. et. al., Arthritis & Rheumatism. 1998; 41(5): 778-799). OA can occur in any joint, but most often affects hands, knees, hips or spine. Risk factors of OA include excessive loading on joints, obesity, heredity, gender (female), trauma, decreased circulation, mal-alignment, and repetitive mechanical stress (occupation, over-exercise). OA may also be the result of free radical damage.

Articular cartilage is a matrix without blood vessel, nerve or lymphatics. The matrix is synthesized by chondrocytes that comprise only 1% of the biomass. Chrondrytes do not divide in adult articular cartilage under normal condition. The extracellulae matrix is composed of fibrillar networks of type II collagen and highly sulfated proteoglycans. Each proteoglycan subunit (aggrecan) contains a protein core attached by hundreds of long chains of sulfated glycosaminoglycans (GAGs), and aggrecans aggregate into huge mass by linking protein to hyaluronic acids. Collagen network provides tensile strength and proteoglycan provides compressive stiffness to the joints.

Clinical manifestations of arthritis include pain, swelling and functional disabilities. X-ray shows thinning of cartilage, narrowing of joint space and changes in the underlying bone. Occurrence of Arthritis is not limited to human; it also occurs in almost every mammal, such as horses, dogs, and guinea pigs.

Lesions of the superficial articular cartilage do not heal and usually progress to the degeneration of the articular surface. Cartilage restoration by surgeries (debridement and lavage, marrow-stimulating technique (microfracture), osteochondral autologous graft transplantation (OATS) and autologous chondrocyte implantation (ACI)) is in its early stage of development. Total joint replacement is recommended to patients with severe conditions. Intra-articular injection of corticosteroids or hyaluronan, oral administrations of steroids and non-steroidal anti-inflammatory drugs (NSAIDs) are known to reduce symptoms. But these methods cannot halt the underlying cartilage tissue degradation. Several topical agents such as capsaicin have been used for the relief of pain but the relief is only temporary.

Prostaglandins play a major role in the inflammation process. NSAIDs inhibit the production of prostaglandin and thus are extensively used to reduce pain and swelling caused by arthritis. However, long-term use of NSAIDs can lead to gastrointestinal ulcers and renal damage. NSAIDs may even speed up the progression of OA (Rashad S et al., Lancet, 1989; 2: 519-521). An alternative of NSAIDs is corticosteroids, which has even more drastic side effects when long term therapy is conducted.

Cyclooxygenase (COX) is the key enzyme for PG synthesis via arachidonic acid/prostaglandin pathway. There are two isoforms, COX I and COX II (Smith W L. et. al. J. Biol Chem, 1996; 271: 33157-33160). COX I is expressed constitutively in most tissues and is involved in many physiological activities, such as coordinating actions of circulating hormones and regulating vascular homeostasis. COX II is induced in inflammatory processes by factors such as Lipopolysaccharide (LPS) or Interleukine 1β (IL-1β) in synovial fibroblasts. Specific inhibitors of COX II are thought to have less ulceric side effects (Jouzeau J Y. et. al. Drugs 1997; 53(4): 563-582).

5-lipoxygenase (5-LO) is the first enzyme in the pathway leading to leukotriene synthesis (Sala A. et. al. Biochemistry (Masc) 1998; 63: 84-92). Leukotrienes are potent local mediators which influence inflammatory and allergic response, including asthma, rheumatoid arthritis, psoriasis, thrombotic disease, ulcerative colitis, bronchitis, sinusitis, allergic and non-allergic rhinitis, and lupus.

Despite therapeutic and commercial success of COX II inhibitors, their gastrointestinal profile and cardiovascular safety are still under investigation. The withdrawal of Vioxx (refecoxib) due to its increase in cardiovascular risk in September 2004 alarmed the pharmaecutial industry (PharmaLive, Oct. 1, 2004; Nov. 9, 2004). Other approaches such as dual COX-lipoxygenase inhibitors, NO-NSALDs (Pelletier J M., et. al. Ann. Rheum. Dis. 2003; 62: 501-509) are in the development pipeline of pharmaceutical industry.

Agents that may repair, or slow the degradation of articular cartilage have been described as possessing chondroprotective properties. Examples of these agents include: hyaluronic acid, chondroitin, glucosamine sulfate (Da Camara C C. and Dowless G V. Annals of Pharmacotherapy 1998; 32: 580-587), vitamin C, Cupper, vitamin A, alone or in various combinations. Among agents described above, glucosamine showed modest effect, yet far from satisfaction.

Glucosamine, the precursor of GAGs, from exogenous sources (food and supplements) may alleviate the pain and halt the progression of cartilage degradation. Glucosamine absorbed by the gastrointestinal tract is incorporated into plasma proteins and concentrates in the articular cartilage. Oral glucosanine is later incorporated into newly synthesized proteoglycan in the cartilage matrix (Barclay T S. et. al. Ann of Pharmacother. 1998; 32: 574-579. Noyszewski E A, et al, Arthritis & Rheumatism 2001; 44: 1089-1095). A three year, double blinded, placebo cotrolled clinical study of osteoarthritis showed long-term structure-modifying and symptom-modifying effects of glucosamine sulfate (Reginster J Y, et. al. Lancet 2001; 357: 251-256. Bruyere O. et. al. Menopause. 2004, 11(2): 138-143).

However, there is evidence to show that chondrocytes had excessive capacity to form maximal amounts of glucosamine from glucose so that exogenous glucosamine could not stimulate chondroitin sulfate synthesis (Mroz P J, and Silbert J E. Biochem J. 2003;-376(Pt 2): 511-515). It was also shown that 40% of chondrocytes obtained from OA patients failed to respond to glucosamine sulfate (Dodge G R, and Jimenez S A. Osteoarthritis Cartilage. 2003; 11(6): 424-432). Although glucosamine is generally well tolerated, adverse effects of gastrointestinal system, such as heartburn and epigastric pain, reversible systolic hypertension, proteinuria, elevated creatine phosphokinase and asthma exacerbation have been reported (Danao-Camara, T. Arthritis Rheum. 2000; 43(12): 2853. Tallia A F and Cardone D A. J. Am Board Fam Pract. 2002; 15: 481-484).

Matrix metalloproteinases (MMPs) are calcium dependent, zinc containing endopeptidases that degrade extracellular matrix at neutral pH. There are several MMPs, for example, MMP-1, MMP-2, MMP-3, MMP-13 in chondrocyte, and MMP-8, MMP-9 in neutrophil increase in OA condition. Inhibitors targeting broad spectrum or specific MMP, showed efficacy in animal OA models (Yoshibara Y., et. al. Ann Rheum Dis. 2000; 59: 455-461. Catterall J B and Cawston T E. Arthritis Res & Therapy. 2003; 5(1): 12-24). The antibiotic, tetracycline, and its semisynthetic derivatives, doxycycline and minocycline, have modest MMP inhibitory properties and are under clinical investigations in the treatment of both OA and RA.

Reactive oxygen species (ROS), mainly nitric oxide, peroxynitrite and superoxide anion. radicals are implicated in aging of cartilage and in the pathogenesis of osteoarthritis. ROS may cause damage to matrix components, either by oxidative attack or by reducing synthesis, inducing degradation, and inducing apoptosis. LPS induced the release of aggrecan. Antioxidants can influence aggrecan degradation by chondrocyte (Tiku M L, et. al., Free Radic Res. 1999; 30(5): 395-405. Henrotin Y E., et. al., Osteoarthritis Cartilage. 2003; 11(10): 747-755.). Bovine superoxide dismutase (SOD), applied intraarticularly, had been proved to be effective in osteoarthritis of the knee joint in three placebo-controlled and one steroid-controlled double-blind trials (Flohe L. Mol Cell Biochem. 1988; 84(2): 123-131).

The maintenance of articular cartilage requires a balance between anabolic and catabolic processes which involves cytokines including insulin like growth factor I (IGF-I), transforming growth factor-beta (TGF-beta), interleukin-1 (IL-1), other agents (vitamin A, C), and mechanical stresses. IL-1 or mechanical stress induces the synthesis of nitric oxide (NO) by inducible nitric oxide synthase (iNOS) from L-arginine in synoviocytes and chondrocytes.

Normal cartilage does not produce NO or express iNOS unless it is stimulated by cytokines or mechanical stresses. In the joint, NO exerts a number of catabolic effects on chondrocyte functions, including: inhibition of collagen and proteoglycan synthesis, activation of matrix metalloproteinases, increased susceptibility to injury by other oxidants, and apoptosis (St Clair E W. J. of Rheumatology 1998, 25(8): 1451-1453). NO also inhibits the respiration and ATP synthesis of chondrocytes (Tomita M, et al, Arthritis & Rheumatism 2001; 44: 96-104). Patients with osteoarthritis have been shown to have an increased level of NO in their synovial fluid (Henrotin Y E, et al, J of Rheumatology 1998; 25(8): 1595-1601). Inhibiting iNOS, and thus NO production, was able to halt the progression of experimental osteoarthritis in dogs (Pelletier J P, et al. Arthritis & Rheumatism 1998; 41: 1275-1286). Clinically used NSAIDs, such as aspirin, sodium salicylate and tetracycline inhibited the expression of NOS protein (Clancy R M, et al, Arthritis & Rheumatism 1998; 41(7): 1141-1151) in addition to inhibition of COX.

Chondrocytes in three-dimensional (3D) alginate cultures showed increased expression of type II collagen and addition of bone morphogenetic protein-2 (BMP-2) enhanced the increase (Grunder T, et al. Osteoarthritis Cartilage. 2004; 12(7): 559-567). Chondrocytes exposed to human recombinant BMP-2 maintained its expression of type-II collagen and showed increased expression of aggrecan in a long-term monolayer culture. Treating chrondrocytes with BMP-2 did not induce the expression of type-X collagen and osteocalcin, indicating no alteration in phenotype (Sailor L Z, et al. J Orthop Res. 1996; 14(6): 937-945).

Recently, scientists have found that Indian hedgehog (Ihh), a member of the vertebrate hedgehog morphogen family, is a key signaling molecule that controls chondrocyte proliferation and differentiation. Cyclic mechanical stress greatly induces the expression of Ihh that up-regulates BMP2/4 in chondrocytes. (Wu Qiu-qian, et al. J. Biol. Chem. 2001; 276(38): 35290-35296).

The mechanical stress exerted on chondrocyte and cartilage leads to the deformation of cells and changes in hydrostatic pressure. Applynig shear stress of continuous laminar fluid by a cone viscometer has shown to alterate chondrocyte shape, increase glycosaminoglycan side chains, increase release of prostaglandin E₂ and IL-6, increase the production of NO, decrease expressions of COL II and aggrecan in vitro (Mohtai M., et al. J. Orthop Res. 1996; 14: 67-73; Smith R L., et al. J. Orthop Res. 1995; 13: 824-831. Lee M S. et. al., J. of Orthop Res. 2002; 20: 556-561). This in vitro stress model mimics one of the shear stresses exerted on the weight bearing articular joints during daily activity, and could serve as a model for potential drug screening.

### SUMMARY OF THE INVENTION

The present invention provides a composition for preventing or treating diseases or disorders caused by degeneration, and/or inflammation comprising (a) Lactuca sativa L., (b) Aquilaria agallocha Roxb. and (c) Atractylodes macrocephala Koidz.

The present invention also provides a method of preventing or treating diseases or disorders caused by degeneration and/or inflammation comprising administering to a subject in need thereof an effective amount of the composition or extract of (a) Lactuca sativa L., (b) Aquilaria agallocha Roxb. and (c) Atractylodes macrocephala Koidz.

### BRIEF DESCRIPTION OF THE DRAWINGS

FIG. 1 is HPLC fingerprint of the composition of the invention. ODS column, 5 μm, 4.6 mm ID., mobile phase, 0.1% phosphoric acid/acetonitrile=93/7, flow rate: 0.8 ml/minute and using phenylbenzimidazole sulfonic acid as internal standard. The peak at relative retention time 1.68 is chlorogenic acid.

FIG. 2 is micromass culture of chondrocytes in induction medium containing herbal preparations. Cells were grown for two weeks before measuring. From left to right: Lane 1, 2: preparation A-0.01, 0.03 mg/ml; lane 3, 4: preparation B-0.01, 0.03 mg/ml; lane 5, 6: preparation F-0.01, 0.03 mg/ml; lane 7: preparation J-0.01 mg/ml; lane 8: control. Right: Lane 1, 2: preparation G-0.01, 0.03 mg/ml; lane 3, 4: preparation H-0.01, 0.03 mg/ml; lane 5, 6: preparation I-0.01, 0.03 mg/ml; lane 7: glucosamine-1 mM; lane 8: control. Row A, B: alkaline phosphatase (ALP) stained by Naphthol AS-MX phosphate/Fast red; row C, D: collagen stained by Sirius red; row E, F: glycosaminoglycan stained by Safranin O.

FIG. 3 is the synthesis of ALP, collagen and GAGs in chondrocytes treated with herbal preparations in combination with glucosamine. Expression was measured on micromass culture after cells grown for two weeks. Induction medium alone (left); with 0.1 mM glucosamine (middle); with 1 mM glucosamine (right). Lane 1: preparation A, lane 2: preparation B, lane 3: preparation F, lane 4: preparation G, lane 5: preparation H, lane 6: preparation I, lane 7: preparation I-1, lane 8: control. All the preparations were at 0.01 mg/ml. Row A, B: alkaline phosphatase (ALP) stained by Naphthol AS-MX phosphate/Fast red; row C, D: collagen stained by Sirius red; row E, F: glycosaminoglycans stained by Safranin O.

FIG. 4 is stimulation of ALP, collagen and GAGs synthesis in mesenchymal stem cells. Herbal preparations were treated to cells grown in the induction medium in micromass culture for two weeks. From left to right: Lane 1, 2: preparation A-0.01, 0.03 mg/ml; lane 3, 4: preparation B-0.01, 0.03 mg/ml; lane 5, 6: preparation F-0.01, 0.03 mg/ml; lane 7: preparation J-0.01 mg/ml; lane 8: control. Right: Lane 1, 2: preparation G-0.01, 0.03 mg/ml; lane 3, 4: preparation H-0.01, 0.03 mg/ml; lane 5, 6: preparation I-0.01, 0.03 mg/ml; lane 7: glucosamine 1 mM; lane 8: control. Row A, B: alkaline phosphatase (ALP) stained by Naphthol AS-NX phosphate/Fast red; row C, D: collagen stained by Sirius red; row E, F: glycosaminoglycans stained by Safranin O.

FIG. 5 is the production of NO by chrondrocytes pretreated with various herbal preparations for two weeks after fluid induced shear stress. Chondrocytes were treated with various herbal combinations at 0.01 mg/ml or treated with glucosamine at 1 mM in F12/DMEM containing 5% FCS, 6.25 ug/ml bovine insulin, 25 ug/ml ascorbic acid and 100 nM Dexamethasone for 2 weeks with medium changing twice a week.

FIG. 6 is gene expressions of shear-stressed vs. non-shear-stressed chondrocytes. One set of representative experimental data was shown. From left to right: lane1: molecular weight ladder, lane 2: shear-stressed control, lane 3: shear-stressed preparation A, lane 4: shear-stressed preparation B, lane 5: shear-stressed preparation F, lane 6: shear-stressed preparation G, lane 7: shear-stressed preparation H, lane 8: shear-stressed Glucosamine; lane 9 non-shear-stressed control, lane 10: non-shear-stressed preparation A, lane 11: non-shear-stressed preparation B, lane 12: non-shear-stressed preparation F, lane 13: non-shear-stressed preparation G, lane 14: non-shear-stressed preparation H, lane 15: non-shear-stressed Glucosamine.

FIG. 7 is MMP activities of serum free culture supernatants of non-shear-stressed (left) vs. shear-stressed (right) chondrocytes. Lane 1: control, lane 2: preparation A, lane 3: preparation B, lane 4: preparation F, lane 5: preparation G, lane 6: preparation H, lane 7: preparation I, lane 8: glucosamine, lane 9: protein molecular weight markers (118, 86, 47, 34, 26, 19K).

FIG. 8 is TIMP activities of serum free culture supernatants of non-shear-stressed (left) vs. shear-stressed (right) chondrocytes. Lane 1: blank (DMEM), lane 2: control, lane3: preparation A, lane 4: preparation B, lane 5: preparation F, lane 6: preparation G, lane 7: preparation H, lane 8: preparation I, lane 9: glucosamine, lane 10: protein molecular weight markers (118, 86, 47, 34, 26, 19K).

FIG. 9 is gene expressions of bone marrow mesenchymal stem cells. One set of representative experimental data of gene expressions was shown. Monolayer vs. micromass cultures: control, preparation F.

### DETAILED DESCRIPTION OF THE INVENTION

The present invention provides a composition for preventing or treating diseases or disorders caused by degeneration, and/or inflammation comprising (a) Lactuca sativa L., (b) Aquilaria agallocha Roxb., and (c) Atractylodes macrocephala Koidz.

The term “effective concentration” or “effective amount” is used to describe an amount or concentration of an active agent or composition according to the present invention which is used in the present invention to produce an intended result. In the case of the present invention, effective concentrations are generally concentrations which are effective to treat diseases or disorders related to degeneration and/or inflammation. The term effective concentration or amount subsumes the administration of a pharmaceutically active agent according to the present invention for a period consistent with the realization of the intended result.

In the compositions of the invention, the percentage of Lactuca sativa L. is about 5%-20% by weight, the percentage of Aquilaria agallocha Roxb. is about 2%-10% by weight, and the percentage of Atractylodes macrocephala Koidz. is about 1%-6% by weight. Preferably, the percentage of Lactuca sativa L. is about 10% by weight, the percentage of Aquilaria agallocha Roxb. is about 5% by weight, the percentage of Atractylodes macrocephala Koidz. is about 3% by weight.

In the preferred embodiment, the composition of the invention is able to prevent or treat arthritis and/or symptoms associated with arthritis.

The preferred composition has 6 major peaks when subjected to high performance liquid chromatography (HPLC) fingerprint analysis, and the relative retention times of the peaks relative to phenylbenzimidazole sulfonic acid are 0.70, 0.78, 1.24, 1.68, 1.87, and 2.12, wherein the peak at relative retention time 1.68 is chlorogenic acid.

It has been found that the composition of the present invention promotes chondrocyte proliferation, inhibits fluid shear stress induced NO production, inhibits matrix degradation enzyme activities, induces tissue inhibitor of matrix metalloproteinase, increases synthesis of collagen and glycosaminoglycan via induction of BMP-2. The composition of the invention has also been proved to induce the chondrogenesis of bone marrow mesenchymal stem cells. The composition of the invention performed its anti-inflammatory activities by dual inhibition of COX II and 5-LO. The present invention shows anti-oxidant activity and chondroprotective activity by superoxide dismutase mimetic activity. The present invention is thus able to prevent and treat arthritis, to repair or regenerate articular joint surfaces and to relieve symptoms associated with degeneration and inflammation diseases.

Further, the composition of the invention has the following physiological and/or pharmacological functions:
(a) the proliferation of chondrocytes,
(b) the synthesis of collagen and/or glycosaminoglycans in chondrocytes or bone marrow mesenchymal stem cells;
(c) the production of tissue inhibitor of matrix metalloproteinase produced by chondrocytes under fluid induced shear stress;
(d) the inhibition of active or latent form of matrix metalloproteinase 2 produced by chondrocytes under fluid induced shear stress
(e) the expressions of genes selected from the group consisting of type II collagen, aggrecan, aggrecanase and bone morphogenetic protein-2 in chondrocytes under fluid induced shear stress;
(f) the inhibition of NO production by chondrocytes under fluid induced shear stress;
(g) the inhibition of matrix degradation enzyme (such as matrix metalloproteinase 3, matrix metalloproteinase 8 and/or matrix metalloproteinase 13) activity;—
(h) the inhibition of cyclooxygenase II and lipooxgenase 5; and
(i) superoxide dismutase mimetic activity.

A whole plant or a certain part of the herbal plant can be used for the preparation of the pharmaceutical composition. Preferably, seeds of the Lactuca sativa L., resin-containing trunks of the Aquilaria agallocha Roxb., and rhizome of the Atractylodes macrocephala Koidz. are used according to the present invention.

The present invention also provides processes of manufacturing the herbal combinations of the present invention. The herbal combination formulations are prepared by one of the following methods:
(a) water or organic solvent (for example, but not limiting to, alcohol) extract of each individual herb is concentrated and then the concentrated extracts are combined; or
(b) all of the raw materials of the herbs are boiled together in water or organic solvents and then concentrated by spray drying or other known methods into dry powder or granulated forms.

The above herbs are typically dried and ground to a fine powder. The composition is typically an intimate mixture of powders. However, herbal extractions may also be used.

It has been found that herbal combinations of the invention show stimulation of chondrocyte proliferation, enhancement of type II collagen and GAGs synthesis via induction of BMP-2. Particularly the preferred composition, preparation F, showed inhibitory activity on alkaline phosphatase which is highly expressed in chondrocytes undergoing hypertrophy. Therefore, the compositions of the present invention promote the expression of type II collagen and the synthesis of GAGs in chrondrocyte.

In an in vitro model of fluid induced shear-stress exerted by a cone viscometer to primary human monolayer chondrocytes, it has been found that compositions of the invention also inhibited NO production 24 hours after stressing. NO was thought to be the most important mediator of cytokines and mechanical stress to chondrocyte of OA. NO leads to inhibition of type II collagen (Col II) and aggrecan synthesis, activation of MMPs, and thus to the degradation of cartilage. It further was discovered that under shear-stressed condition, compositions of the invention recovered the expression of Col II back to the non-shear-stressed control level. It has been found that the activity of MMP-2 (both the 68K active form and 72K latent form) was inhibited by compositions of the present invention under shear stressed condition.

Therefore, compositions of the present invention inhibit the NO production, resume the synthesis of Col II, aggrecan, and aggrecanase and inhibit MMPs.

Increases in aggrecan, and aggrecanase expression indicated an increase in synthesis and remodeling of GAGs that resulted in higher accumulation of GAGs evidenced by higher Safranin O staining of micromass cultured chondrocytes. It was well documented that chondrocytes in three-dimensional (3D) alginate cultures raised type II collagen expression significantly and addition of BMP-2 enhanced this effect (Grunder T. et al., Osteoarthritis Cartilage. 2004; 12(7): 559-567).

Under non-shear-stressed condition, glucosamine inhibited the expression of aggrecan significantly. The expressions of aggrecanase were parallel with the expression of aggrecan and BMP-2 in shear-stressed and non-shear-stressed conditions by pretreatment of various herbal preparations and glucosamine.

The expression of BMP-2 was down-regulated by this mechanical stress condition when compared with non-shear stressed control. However, it has been found that preparation B and F of the invention were effective in inducing BMP-2 expression under shear stressed condition. Therefore, compositions of the invention induce expression of BMP-2.

It has been discovered that under shear-stressed condition, compositions of the invention stimulated TIMP. Stimulation of TIMP appeared to also contribute to the accumulation of collagen and glycosaminoglycan in extracellular matrix.

It also been found that compositions of the invention also inhibited COX II and 5-LO, key enzymes that lead to prostaglandins and leukotrienes synthesis. Prostaglandins and leukotrienes are mediators for inflammations. The dual inhibitions of these inflammatory mediators could help to alleviate RA and OA symptoms such as pain and swelling, without deastic side effects.

The response of OA chondrocytes to shear-stress was modulated by the herbal preparations of the present invention to synthesize more Col II and aggrecan for repairing and regenerating hyaline cartilage via induction of BMP-2 and TIMP, and inhibition of NO production and MMPs activity, and protect cartilage from ROS by SOD mimetic activity.

It appeared that herbal preparations in the present invention induced BMP-2 expression under fluid induced shear stress condition, and led to the expressions of Col II, aggrecan, and aggrecanase. Higher expression of Col II accompanied by less MMP-2 (both active and latent form), MMP-3, MMP-8, and MMP-13 activities led to accumulation of collagen and glycosaminoglycan by the present invention. Under shear-stressed condition, the compositions of the invention stimulated TIMP. Stimulation of TIMP appeared to also contribute to the accumulation of collagen and glycosaminoglycan in extracellular matrix. Increases in aggrecan and aggrecanase expression indicated an increase in synthesis and remodeling of GAGs that resulted in higher accumulation of GAGs as evidenced by higher Safranin O staining of micromass cultured chondrocytes.

It was well documented that chondrocytes in three-dimensional (3D) alginate cultures raised type II collagen expression significantly and addition of BMP-2 enhanced this effect (Grunder T. et al., Osteoarthritis Cartilage. 2004; 12(7): 559-567). The response of OA chondrocytes to shear-stress was modulated by the compositions of the present invention to synthesize more Col II and aggrecan for repairing and regenerating hyaline cartilage via induction of BMP-2 and TIMP, and inhibition of NO production and MMPs activity. AG-041R, a novel indolin-2-one derivative, accelerated proteoglycan synthesis and up-regulated the expression of type II collagen, aggrecan and BMP-2 genes. In contrast, AG-041R suppressed ALP activity indicating that AG-041R prevented chondrocyte terminal differentiation. (Okazaki M, Osteoarthritis Cartilage. 2003; 11(2): 122-132). Preparation F showed similar biochemical profile as AG-041R. It stimulated Col II, aggrecan, aggrecanase, TIMP and BMP-2 expression, inhibited NO production, and inhibited MMPs and ALP activity that prevented terminal differentiation of chondrocytes under fluid induced shear stress.

It also induced the differentiation of bone marrow mesenchymal stem cells into chondrogenic lineage. Combination of low dose glucosamine with the compositions of the present invention showed additive effect on stimulation of collagen and glycosaminoglycan synthesis. The compositions of the present invention also inhibit MMP-3, 8, 13 that are considered most important factors involving in cartilage degradation. The compositions of the present invention have SOD mimetic activity to combat the ROS produced during aging and pathological processes of arthritis.

In summary of the biological activity of the present invention, the compositions of the present invention showed effect on stimulation of collagen and glycosaminoglycan synthesis. The dual inhibitory activity on COX II and 5-LO of compositions of the invention also ensured its activity on controlling pain and inflammation of arthritis.

Further, it has been found that in human use experiences, the compositions of the present invention decreased 60% of pain in lower back, pain in hip and knee, and decreased difficulties in carrying daily living tasks without suffering side effects.

By stimulating the proliferation of chondrocytes, increasing the synthesis of type II collagen and glycosaninoglycan in chondrocytes, inducing the differentiation of bone marrow mesenchymal stem cells, stimulating TIMP, inhibiting MMP-2, 3, 8, 13 and NO production, dual inhibiting COX II and 5-LO, and SOD mimetic activity, compositions of the present invention can be used to prevent and treat arthritis, repair and regenerate articular joint surfaces and relieve symptoms associated with arthritis in humans and animals.

In addition to herbs above, various pharmaceutically acceptable additives, carriers, diluents, excipients and/or fillers may be present in the composition. The formulations can be prepared in any desirable medicine dosage forms, such as a lozenge, tablet, film coated tablet, capsule, soft capsule, granule, powder, pill, solution, emulsion, injection solution, injection, ointment, cream, spray, inhalant, soft gel, liquid, honey ball, lotion for oral, injection, topical, tansmucosal or transdermal administrations.

The present composition and extracts of composition may also be employed either alone or in combination with other compounds as a part of combination therapy, partially or completely, in place of other conventional anti-inflammatories and chondroprotectives, such as together with steroids, NSAIDs, 5-lipoxygenase inhibitors, leukotriene antagonists, LTA4 hydrolase inhibitors, and LTC4 synthase inhibitors. One will typically combine a drug or drugs and a nutraceutical, such as herbal combinations of the current invention, in a manner such that the drug and the nutraceutical have different mechanisms of action, but yet target the same disease. For example, in a typical selection of agents for use in combination therapy to treat arthritis, one could utilize the herbal compositions of the present invention, with another agent known to attenuate inflammation associated with arthritis via an independent mechanism. The most preferred nutraceutical is glucosamine.

The present invention further provides a method of preventing or treating diseases or disorders caused by degeneration and/or inflammation comprising administering to a subject in need thereof an effective amount of the composition or extract of (a) Lactuca sativa L., (b) Aquilaria agallocha Roxb., and (c) Atractylodes macrocephala Koidz.

The extract or the compositions of the invention is administered by oral, subcutaneous injection, intra-muscular injection, intra-venous injection, intra-articular injection, mucosal membrane or topical routes.

The extract or the compositions of the invention could be administered in combination with a second therapeutic agent such as those selected from the group consisting of glucosamine, glucosamine sulfate, chondroitin sulfate, prednisone, dexamethasone, beclosmethasone, methylprednisone, betamethasone, hydrocortisone, methotrexate, cyclosporin, rapamycin, tacrolimus, antihistamine drugs, TNF antibodies, IL-1 antibodies, soluble TNF receptors, soluble IL-1 receptors, TNF or IL-1 receptor antagonists, non-steroidal anti-inflammatory agents, COX-2 inhibitors.

In particular, the extract or the compositions of the invention used in the method of the invention could be applied to
(a) repair or regenerate articular joint surfaces;
(b) have chondroprotective activities;
(c) stimulate the proliferation of chondrocytes;
(d) synthesize collagen and/or glycosaminoglycans in chondrocytes or bone marrow mesenchymal stem cells;
(e) inhibit nitric oxide production in chondrocytes under fluid induced shear stress;
(f) inhibit active or latent form of matrix metalloproteinase 2 produced by chondrocytes under fluid induced shear stress;
(g) stimulate tissue inhibitor of matrix metalloproteinase production by chondrocytes under fluid induced shear stress;
(h) stimulate the expression of genes selected from the group consisting of type II collagen, aggrecan, aggrecanase and bone morphogenetic protein-2 in chondrocytes under fluid induced shear stress;
(i) inhibit matrix metalloproteinase-3, 8, 13;
(j) inhibit cyclooxygenase II;
(k) inhibit lipooxgenase 5;
(l) have superoxide dismutase mimetic activity; and
a combination thereof.

### EXAMPLES

Herbal Combination Manufacturing Processes:

The herbal combination formulations were manufactured by one of the following methods:
(a) water or organic solvent (for example, but not limiting to, alcohol) extract of each individual herb was concentrated and then the concentrated extracts were combined; and
(b) all of the raw materials of the herbs were boiled together in water or organic solvents and then concentrated by spray drying or other known methods into dry powder or granulated forms.

The formulations could be prepared in many desirable medicine dosage forms, such as a tablet, soft gel, granule, capsule, liquid, honey ball, cream, lotion or ointment for oral, injection, topical, tansmucosal or transdermal administrations.

### Example 1

### Herbal Formulation Examples

| | | |
|---|---|---|
| Ingredient | % by weight | Solvent for extraction |

Formulation A

| | | |
|---|---|---|
| Lactuca sativa L. | 5% | water |
| Aquilaria agallocha Roxb. | 4% | water |
| Atractylodes macrocephala Koidz. | 1% | water |

Formulation B

| | | |
|---|---|---|
| Lactuca sativa L. | 15% | alcohol |
| Aquilaria agallocha Roxb. | 6% | alcohol |
| Atractylodes macrocephala Koidz. | 5% | alcohol |

Formulation F

| | | |
|---|---|---|
| Lactuca sativa L. | 10% | 50% alcohol |
| Aquilaria agallocha Roxb. | 5% | 50% alcohol |
| Atractylodes macrocephala Koidz. | 3% | 50% alcohol |

Formulation G

| | | |
|---|---|---|
| Lactuca sativa L. | 15% | 50% alcohol |
| Aquilaria agallocha Roxb. | 10% | 50% alcohol |
| Atractylodes macrocephala Koidz. | 6% | 50% alcohol |

Formulation H

| | | |
|---|---|---|
| Lactuca sativa L. | 20% | water |
| Aquilaria agallocha Roxb. | 8% | water |
| Atractylodes macrocephala Koidz. | 5% | water |

Formulation I

| | | |
|---|---|---|
| Lactuca sativa L. | 10% | 50% alcohol |
| Aquilaria agallocha Roxb. | 3% | 50% alcohol |
| Atractylodes macrocephala Koidz. | 1% | 50% alcohol |

Formulation J

| | | |
|---|---|---|
| Lactuca sativa L. | 5% | alcohol |
| Aquilaria agallocha Roxb. | 2% | alcohol |
| Atractylodes macrocephala Koidz. | 1% | alcohol |

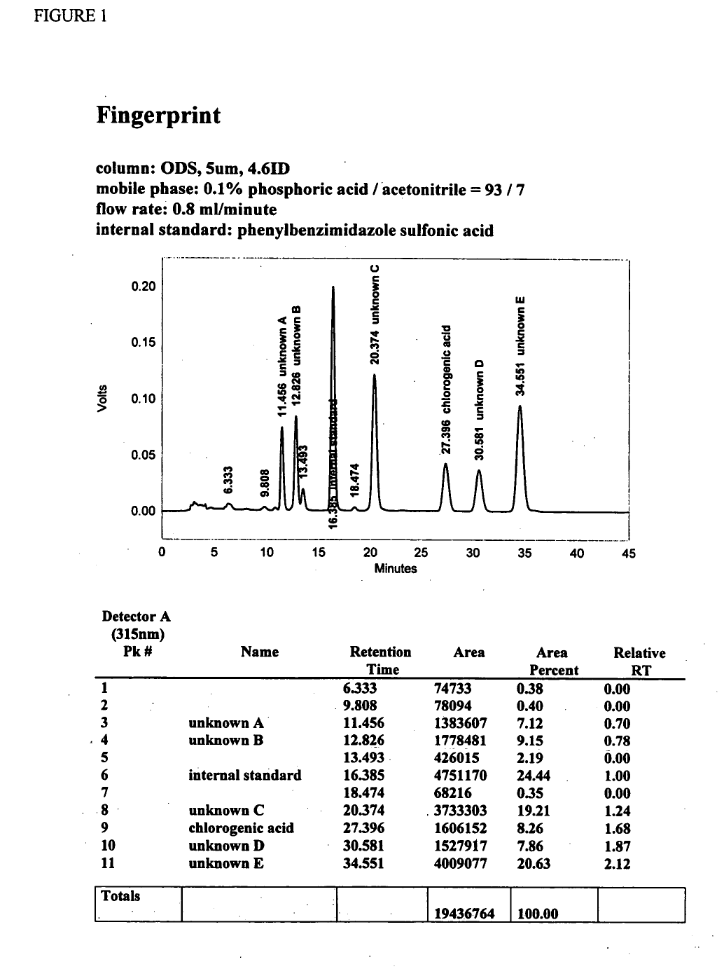

The high performance liquid chromatography (HPLC) fingerprint of formulation F is shown in FIG. 1 (FIG. 1).

When formulation F of the invention was subjected to HPLC analysis, a fingerprint profile of 6 major peaks was found as shown in FIG. 1. Data of the peaks is tabulated in Table 1. The retention times of the 6 major peaks relative to the internal standard (phenylbenzimidazole sulfonic acid) were 0.70, 0.78, 1.24, 1.68, 1.87, and 2.12, respectively. The peak at relative retention time 1.68 was chlorogenic acid.

TABLE 1
Major peaks of example F by HPLC

| Peak No. | Peak Name | Retention time | Relative Retention Time |
|---|---|---|---|
| 3 | Unknown A | 11.456 | 0.70 |
| 4 | Unknown B | 12.826 | 0.78 |
| 6 | Internal Standard | 16.385 | 1.00 |
| 8 | Unknown C | 20.374 | 1.24 |
| 9 | Chlorogenic Acid | 27.396 | 1.68 |
| 10 | Unknown D | 30.581 | 1.87 |
| 11 | Unknown E | 34.551 | 2.12 |

### Example 2

### Preparation of Cells

Human Articular Chondrocyte

Human articular cartilage was obtained from patients who received total joint replacement with consent. The cartilage was digested with 1 mg/ml of Class 2 and Class 4 bacterial collagenase (Worthington Chemical, Freehold, N.J.) in DMEM containing 10% fetal calf serum (FCS) and 25 ug/ml Gentamicin (Gibco, Grand Island, N.Y.) (C-DMEM) at 37° C. overnight. The primary cells were plated at high density in 10 mm culture dish (Nunc, Roskilde, Danmark) in C-DMEM and cultured under humidified atmosphere containing 5% CO₂ at 37° C.

Human Bone Marrow Mesenchymal Stem Cell:

Human bone marrow mesenchymal stem cells were obtained from patients receiving total joint replacement with consent. The cells were cultured in DMEM containing 10% fetal calf serum (FCS), and 25 ug/ml Gentamicin (Gibco, Grand Island, N.Y.) (C-DMEM) at 37° C. under humidified atmosphere containing 5% CO₂.

### Example 3

### Cell Proliferation Assay.

1×10⁴ cells in 100 ul of C-DMEM were plated into each well of 96 multiwell dishes (Nunc, Roskilde, Danmark). After 48 hours, the media were replaced with fresh C-DMEM containing designated concentrations of herbal combinations. 72 hours later, the media were aspirated, 50 ul of MTT (0.5 mg/ml in PBS) was added, and cells were incubated at 37° C. in humidified atmosphere containing 5% CO₂ for 4 hours. At the end of the incubation, solutions were aspirated, and the reduced formasan was dissolved in 100 ul of isopropanol containing 0.04 N HCl. The absorbance at 570 nm was measured by an ELISA reader (Molecular Device).

As shown in Table 2, Preparation B, F, H stimulated chondrocyte proliferation in a dose dependent manner, while preparation A did not show significant effect. Preparation A and F were toxic to the cells at 0.1 mg/ml.

TABLE 2
Stimulation of chondrocyte proliferation

| | A | | B | | F | | H | |
|---|---|---|---|---|---|---|---|---|
| ConC. | | % change | | % change | | % change | | % change |
| (mg/ml) | mean OD | of control | mean OD | of control | mean OD | of control | mean OD | of control |
| blank | 0.040 | | 0.040 | | 0.040 | | 0.040 | |
| 0 | 0.174 | | 0.174 | | 0.174 | | 0.174 | |
| 0.1 | 0.173 | −9 | 0.239 | 36 | 0.129 | −39 | 0.239 | 48 |
| 0.05 | 0.188 | 1 | 0.238 | 35 | 0.286 | 68 | 0.240 | 49 |
| 0.025 | 0.197 | 7 | 0.212 | 17 | 0.253 | 45 | 0.227 | 39 |
| 0.0125 | 0.189 | 1 | 0.211 | 16 | 0.228 | 28 | 0.215 | 30 |
| 0.0062 | 0.191 | 3 | 0.210 | 16 | 0.226 | 27 | 0.212 | 28 |

### Example 4

### Micromass Culture System

Ten ul of 1×10⁷ cells in C-DMEM was seeded into the center of each well of 48 multiwell culture dish (Nunc, Roskilde, Danmark). Two hours later, 250 ul of F12/DMEM containing 5% FCS, 6.25 ug/ml bovine insulin, 25 ug/ml ascorbic acid and 100 nM Dexamethasone (induction medium) with various concentrations of herbal combinations or glucosamine (Sigma) was added. The media were changed twice a week for two weeks. At the end of the incubation, the media were changed with fresh DMEM. Twenty-four hours later, the media were collected for MMP zymography study and cells were fixed and stained for alkaline phosphatase (ALP), collagen or glycosaminoglycan using Naphthol AS-MX phosphate/Fast red, Sirius red, or Safranin O (Sigma), respectively.

A. Stimulation of Collagen and GAGs Synthesis of Chondrocytes

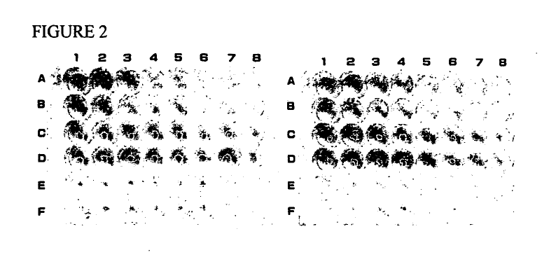

FIG. 2 illustrated micromass culture of chondrocyte in induction containing herbal preparations for two weeks.

As shown in Table 3, at 0.03 mg/ml, preparation F and H stimulated the synthesis of collagen and GAGs as compared to the control. Furthermore, preparation F also inhibited alkaline phosphatase activity. Under the same experimental conditions, glucosamine at 1 mM did not show effects on ALP, collagen or GAGs synthesis.

TABLE 3
Stimulation of ALP, collagen and GAGs synthesis in chondrocytes
by herbal preparations in micromass culture

| | A | | B | | F | | G | | H | | I | | J | Glu | Cnt |
|---|---|---|---|---|---|---|---|---|---|---|---|---|---|---|---|
| | Conc.(mg/ml) | | | | | | | | | | | | | | |
| | 0.01 | 0.03 | 0.01 | 0.03 | 0.01 | 0.03 | 0.01 | 0.03 | 0.01 | 0.03 | 0.01 | 0.03 | 0.01 | 1 mM | 0 |
| ALP | +++ | +++ | +++ | ++ | +++ | + | +++ | +++ | +++ | +++ | +++ | +++ | +++ | +++ | +++ |
| Collagen | + | + | + | + | + | ++ | + | + | + | ++ | + | + | + | + | + |
| GAGs | + | + | + | + | + | ++ | + | + | + | ++ | + | + | + | + | + |

B. Stimulation of ALP, Collagen and GAGs Synthesis in Chondrocytes by Herbal Preparations in Combination with Glucosamine in Micromass Culture

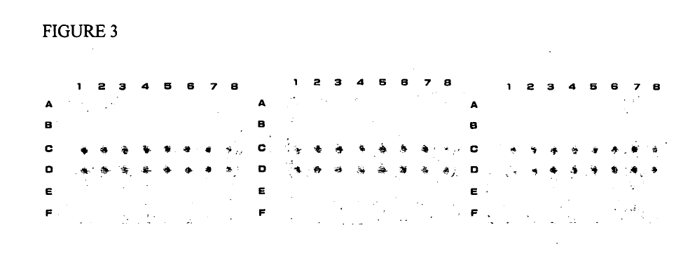

As shown in FIG. 3, at 0.01 mg/ml, preparation F and H stimulated the synthesis of collagen and GAGs as compared to the control. Furthermore, preparation F also inhibited alkaline phosphatase activity. Glucosamine at 0.1 or 1 mM did not affect the activity of herbal preparations. However, it appeared that 0.1 mM glucosamine slightly increased baseline collagen and GAG synthesis. There may be slight additive effect of glucosamine with herbal preparations.

C. Stimulation of ALP, Collagen and GAGs Synthesis in Bone Marrow Mesenchymal Stem Cells by Herbal Preparations in Micromass Culture

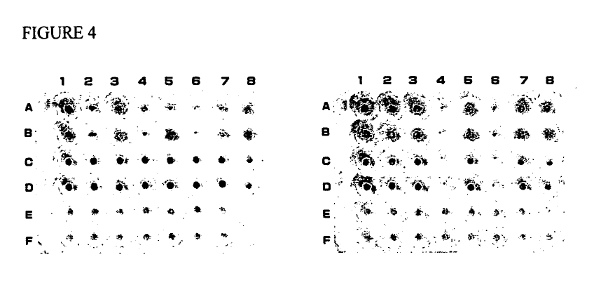

As shown in FIG. 4, preparation F and H stimulated the synthesis of collagen and GAGs in bone marrow mesenchymal stem cells in the induction medium dose-dependently as compared to the control. Furthermore, at 0.03 mg/ml, preparation F, H, I, B, A, in a descending order, inhibited alkaline phosphatase activity. Under the same experimental conditions, glucosamine at 1 mM did not show significant effects on ALP, collagen or GAGs synthesis. Preparation F, H, and I at 0.03 mg/ml induced mesenchymal stem cell mass condensation.

### Example 5

### Fluid Induced Shear Stress Experiments

Confluent cells were treated with various herbal combinations in F12/DMEM containing 5% FCS, 6.25 ug/ml bovine insulin, 25 ug/ml ascorbic acid and 100 nM Dexamethasone for 2 weeks with medium changed twice a week. At the end of each treatment, cells were washed with PBS and incubated in DMEM overnight. Cells were then exposed to fluid-induced shear stress by a custom-made cone viscometer at the rate of 100 rpm for 2 hours. The shear stress induced at this speed was 0.82 Pa (8.2 dyn/cm²). Aliquots of culture medium were collected at designated time for nitric oxide measurement and MMP zymography analysis. Another set of cells was treated similarly without shearing as the control. The cells were washed with PBS, and RNA was extracted by Tri-Reagent (Sigma).

### Example 6

### Nitric Oxide Determination

NO was measured by Griess reagent using sodium nitrite (Sigma) as the standard control. Fifty ul of culture medium was incubated with 25 ul of 1% sulfanilamide in 5% phosphoric acid and 25 ul of 0.1% N-1-naphthyl-ethylenediamine dihydrochloride (Sigma) for 10 minutes at room temperature. Absorbance at 570 nm was measured by an ELISA reader (Molecular device).

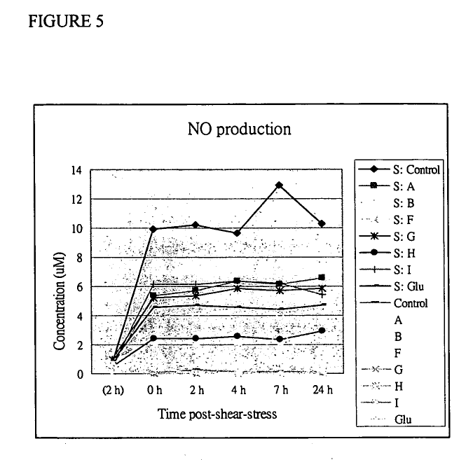

As shown in FIG. 5 and Table 4, two hours of fluid induced shear stress at 100 rpm (0.82 Pa) induced NO production to 10 uM at 0 hour and plateau thereafter to 24 hours. Various herbal preparation treatments inhibited the NO productions. Preparation B and H were the most potent ones that inhibited NO production by 80%, preparation F and glucosamine inhibited 55%, preparation A, G and I inhibited 40%. Non-shear-stressed cells did not produce significant amounts of NO. Preparation B and I in non-sheared cells at 7 hours post-shearing showed some NO production. That may be due to some cell debris that was not removed.

TABLE 4
NO production after shear-stressed of chondrocytes pretreated
with various herbal preparations for two weeks

| | | NO Concentration (uM) | | | | | |
|---|---|---|---|---|---|---|---|
| | | (2 h) | 0 h | 2 h | 4 h | 7 h | 24 h |
| Sheared | S: Control | 0.74 | 9.93 | 10.22 | 9.67 | 12.96 | 10.29 |
| | S: A | 0.51 | 5.33 | 5.72 | 6.38 | 6.12 | 6.57 |
| | S: B | 0.51 | 2.22 | 2.22 | 2.22 | 2.10 | 2.26 |
| | S: F | 0.85 | 3.92 | 4.25 | 4.58 | 4.25 | 4.44 |
| | S: G | 0.97 | 5.20 | 5.33 | 5.86 | 5.72 | 5.86 |
| | S: H | 0.51 | 2.44 | 2.44 | 2.58 | 2.33 | 2.92 |
| | S: I | 0.51 | 6.12 | 6.12 | 6.38 | 6.25 | 5.43 |
| | S: Glu | 0.85 | 4.58 | 4.75 | 4.58 | 4.42 | 4.72 |
| Non-sheared | Control | 0.63 | 0.06 | 0.28 | 0.17 | 0.17 | 0.12 |
| | A | 0.63 | 0.00 | 0.00 | 0.00 | 0.00 | 0.00 |
| | B | 0.63 | 0.00 | 0.00 | 0.06 | 1.19 | 0.00 |
| | F | 0.63 | 0.00 | 0.00 | 0.00 | 0.00 | 0.00 |
| | G | 0.63 | 0.06 | 0.00 | 0.00 | 0.40 | 0.00 |
| | H | 0.28 | 0.00 | 0.00 | 0.00 | 0.00 | 0.00 |
| | I | 0.17 | 0.00 | 0.00 | 0.00 | 2.92 | 0.00 |
| | Glu | 0.17 | 0.40 | 0.00 | 0.00 | 0.00 | 0.00 |

### Example 7

### Reverse Transcription and PCR Amplification

The RNA of cells was extracted with Tri-Reagent (Sigma). 800 ng of total RNA was reverse transcribed by Omniscript reverse transcriptase (Qiagen, Valencia, Calif.) in buffer containing 1 uM random primer, 0.5 mM each of dNTP, and 10 units of ribonuclease inhibitor. Reverse transcription was carried out at 37° C. for one hour by a programmable thermal controller (PTC-100, MJ Research, Inc., Watertown, Mass.). Reaction was terminated by raising the temperature to 93° C. for five minutes.

Polymerase chain reaction (PCR) amplification was performed with primer pairs as listed in Table 5. PCR was performed in a thermal controller (PTC-100, MJ Research, Inc., Watertown, Mass.) with master mix (HotStarTaq DNA polymerase, Qiagen) at initial denaturation at 95° C. for 15 minutes, followed by 25 cycles of 94° C. for 1 minute, 58° C. for 40 seconds, and 72° C. for 40 seconds. The final cycle extended 72° C. for 10 minutes. The PCR products accompanied by 100 bp ET marker were separated by 1.2% agarose gel, stained with ethidium bromide and visualized under UV light. A Polaroid picture was taken by Foto/Phoresis UV documentation system (Fotodyne Inc.). Beta-actin expression under the same experimental conditions was used as a reference to normalize the expressions.

TABLE 5

Primer pairs

| | |
|---|---|
| Gene | Primer Pair 5′-3′ |

| | |
|---|---|
| iNOS | GTG AGG ATC AAA AAC TGG GG |
| | ACC TGC AGG TTG GAC CAC |
| Col I | CGTGGTGACAAGGGTGAGAC |
| | TAGGTGATGTTCTGGGAGGC |
| Col II | CTG GCT CCC AAC ACT GCC AAC GTC |
| | TCC TTT GGG TTT GCA ACG GAT TGT |
| Aggrecan | CAC TGT TAC CGC CAC TTC CC |
| | GAG ATC GTT CCT CTC GCC CT |
| Aggrecanase | AGA TCC AGG AAA GGA GGG CT |
| | AAA GCT CTC TGT AGC CT |
| MMP-2 | GAT GCC TGG AAT GCC ATC CCC GAT |
| | AGG CTT GGT GGG ATT GGA GGG GGA |
| MMP-9 | GAG GTT CGA CGT GAA GGC GCA GAT G |
| | CAT AGG TCA CGT AGC CCA CTT GGT C |
| BMP-2 | CAGAGACCCACCCCCAGCA |
| | CTGTTTGTGTTTGGCTTGAC |
| β-Actin | CAG GTC ATC ACY ATY GGC AAT GAG C |
| | CGG ATG TCM ACG TCA CAC TTC ATG A |

### Example 8

### Gene Expressions of Shear-Stressed vs. Non-Shear-Stressed Chondrocytes

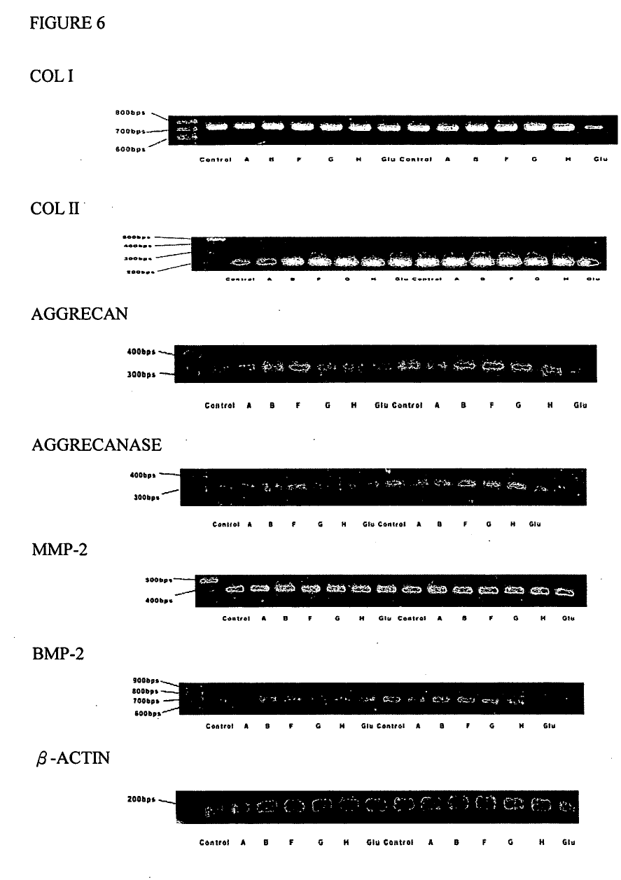

One set of representative experimental data was shown in FIG. 6. From left to right: lane1: molecular weight lader, lane 2: shear-stressed control, lane 3: shear-stressed preparation A, lane 4: shear-stressed preparation B, lane 5: shear-stressed preparation F, lane 6: shear-stressed preparation Q lane 7: shear-stressed preparation H, lane 8: shear-stressed Glucosamine; lane 9 non-shear-stressed control, lane 10: non-shear-stressed preparation A, lane 11: non-shear-stressed preparation B, lane 12: non-shear-stressed preparation F, lane 13: non-shear-stressed preparation G; lane 14: non-shear-stressed preparation H, lane 15: non-shear-stressed Glucosamine.

Col I: Herbal preparation A showed slight inhibition, and preparation B and F showed stimulation on Col I expression under both non-sheared and sheared conditions. However, glucosamine at 1 mM under non-shear-stressed condition inhibited Col I expression significantly, and had no effect under sheared condition.

Col II: Col II expression was down-regulated by two hours of 100 rpm fluid-induced-shear-stress when compared with non-shear stressed control. All the herbal preparations except preparation A counteracted the down-regulation of shear-stress, and brought the Col II expression back to the level of non-sheared control. Glucosamine also showed similar effect as the herbal preparations under shear-stress.

Aggrecan: Under non-shear-stressed condition, herbal preparation B and F significantly enhanced the expression of aggrecan, while glucosamine inhibited the expression of aggrecan. Shear-stress significantly inhibited the expression of aggrecan, however, preparation B and F brought the expression of aggrecan to that of the non-shear-stressed control and glucosamine slightly increased aggrecan expression under shear-stressed condition.

Aggrecanase: The changes of aggrecanase expression followed the same trends of aggrecan expression. Shear-stress inhibited aggrecanase expression, and preparation B and F brought the expression of aggrecan to that of the non-shear-stressed control. However, glucosamine inhibited aggrecanase expression under non-shear-stressed condition, and it showed slight increase in aggrecanase expression under shear-stressed condition.

MMP-2: MMP-2 expression appeared not to be affected by various herbal preparations, glucosamine or shear-stress significantly.

iNOS: iNOS expression was not detected 24 hours after flow-induced-shear stress or non-shear stress condition. iNOS expression was peaked at 6 hours after IL-1 treatment. NO produced was negatively feedback to the induction of iNOS expression in rat vascular smooth muscle cells (Katuyama K et. al. Arterioscler Thromb. Vasc. Biol. 1998; 18(11): 1796-1802).

BMP-2: Under non-sheared condition, preparation A and H slightly inhibited BMP-2 expression, while glucosamine inhibited its expression completely. The expression of BMP-2 was down-regulated by shear-stress, and preparation B and F prevented the down-regulation significantly. Preparation G. H, and glucosamine showed less activity.

β-Actin: The expressions of house keeping gene β-actin was similar in all treatments.

### Example 9

### MMP-2 Activities of Non-Shear-Stressed vs. Shear-Stressed Chondrocytes by Zymography

Equal amount of protein of each culture medium was mixed with equal volume of 2× non-reducing SDS sample buffer, and incubated at room temperature for two hours for sample to denature. The samples were loaded into each well of a 10% SDS polyacrylamide gel containing 1 mg/ml of bovine gelatin (Sigma), and run at 20 mA in ice bath by a BioRad Mini Protean gel apparatus. After dye front reached the bottom of the gel, the gel was washed three times, 15 minutes each, with 2.5% Triton X-100. Then, the gel was renatured in developing buffer (50 mM Tris-HCl, pH7.6, 10 mM CaCl₂, 50 mM NaCl, 0.05% Brij35) at 37° C. for 48 hours. The gel was stained with 0.1% Coomassie blue (Sigma) in 40% methanol and 10% acetic acid, and destained in 30% methanol and 7% acetic acid. The enzyme digested clear bands were visualized in the blue background.

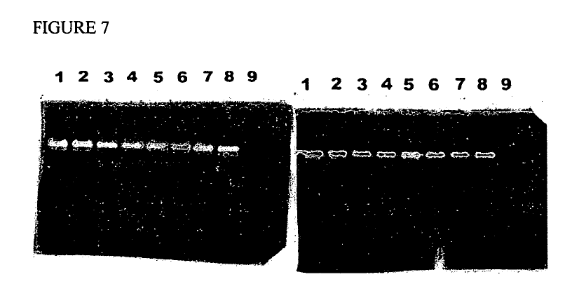

As shown in FIG. 7, under both non-sheared (left) and sheared (right) conditions, the active and latent forms of MMP-2 (the light fine band at 68 K, the major band at 72 K, respectively) were less by treatments of preparation F and H; glucosamine, on the other hand, showed same profiles as controls under both non-sheared and sheared conditions.

### Example 10

### TIMP Activities of Non-Shear-Stressed vs. Shear-Stressed Chondrocytes by Reverse Zymography

Equal amount of protein of each culture medium was mixed with equal volume of 2× non-reducing SDS sample buffer, and incubated at room temperature for two hours for sample to denature. The samples were loaded into each well of a 10% SDS polyacrylamide gel containing 1 mg/ml of bovine gelatin (Sigma) and 1 ml serum free 24 hour's culture supernatant of human foreskin dermal fibroblast which expressed MMP, and run at 20 mA in ice bath by a BioRad Mini Protean gel apparatus. After dye front reached the bottom of the gel, the gel was washed three times, 15 minutes each, with 2.5% Triton X-100. Then, the gel was renatured in developing buffer (50 mM Tris-HCl, pH7.6, 10 mM CaCl₂, 50 mM NaCl, 0.05% Brij35) at 37° C. for 48 hours. The gel was stained with 0.1% Coomassie blue (Sigma) in 40% methanol and 10% acetic acid, and destained in 30% methanol and 7% acetic acid. The enzyme inhibited blue bands were visualized in the light blue background.

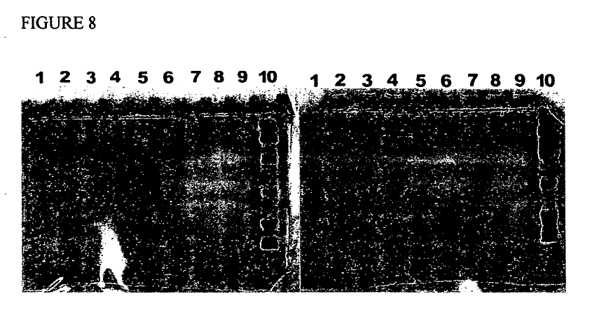

As shown in FIG. 8, under non-sheared condition, there were decrease of TIMP (41 K band, >118 K band: TIMP complexed with MMP) by preparation B, F, G. H, I, Glucosamine and no significant difference in TIMP by preparation A; however, under sheared condition, preparation B, F, G, H increased the TIMP. The molecular weight of TIMP shown in this experiment was larger than the reported TIMPs (21 K to 29 K). The nature of this 41 K TIMP was unclear.

### Example 11

### Gene Expressions of Bone Marrow Mesenchymal Stem Cells

Human bone marrow stem cells were cultured as monolayer to confluency in 10 cm dish or micromass (10 spots of 1×10⁵ cells/10 ul spot) as described above in chondrocytes. The cells were then treated in F12/DMEM containing 5% FCS, 6.25 ug/ml bovine insulin, 25 ug/ml ascorbic acid and 100 nM Dexamethasone in the absence or presence of preparation F at 0.01 mg/ml for 2 weeks with medium changed twice a week. At the end of treatment, the cells were harvested with Tri-Reagent and RT/PCR was performed as in chondrocytes.

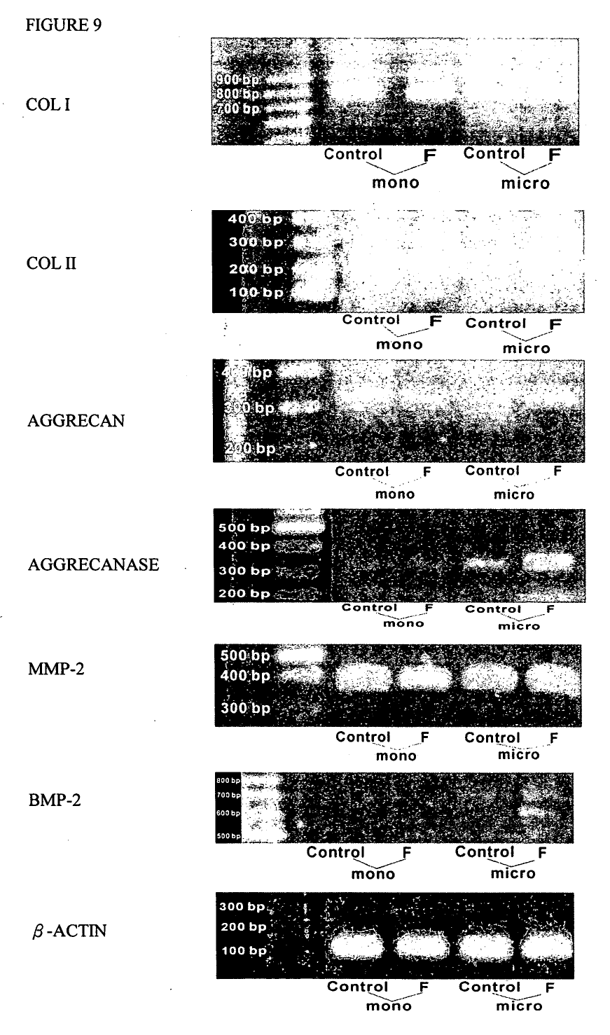

One set of representative experimental data of gene expressions was shown in FIG. 9. Gene expressions were grouped into monolayer and micromass cultures, control and preparation F.

Col I: Monolayer: no difference on the expression of Col I; micromass: no difference on the expression on Col I.

Col II: Monolayer: no difference on the expression of Col II; micromass: no difference on the expression on Col II.

Aggrecan: Monolayer: preparation F stimulated the expression of aggrecan; micromass: preparation F stimulated the expression of aggrecan. Micromass culture treated by preparation F induced the expression of aggrecan more than monolyer culture.

Aggrecanase: Monolayer: preparation F slightly stimulated the expression of aggrecanase; micromass: preparation F stimulated the expression of aggrecanase significantly.

MMP-2: Preparation F did not show effect on the expression of MMP-2 in either monolayer or micromass culture.

BMP-2: Under this experimental condition, only preparation F treatment in micromass cultured mesenchymal stem cells showed slight expression of BMP-2.

β-Actin: The expression of house keeping gene β-actin was similar in all treatments

### Example 12

### COX II Inhibition

Human recombinant COX II expressed in Sf21 cells (Sigma, C-0858) was used. Herbal preparation F or vehicle was preincubated with 0.11 U enzyme, 1 mM reduced glutathione, 500 μM phenol and 1 μM hematin in Tris-HCl, pH 7.7 at 37° C. for 15 minutes. 0.3 μM arachidonic acid as substrate was added and the reaction was terminated by addition of 1 N HCl after 5 minutes. The converted PGE₂ was measured by an Amersham EIA kit following centrifugation. Composition F was assayed at 10 and 100 μg/ml.

As shown in Table 6, IC50 for preparation F was about 10 μg/ml. The IC50 for standard COX II inhibitor, Nimesulide, was 1 μM.

TABLE 6
Inhibition of COX II by preparation F

| | Concentration | | % Inhibition |
|---|---|---|---|
| Preparation F | 10 | μg/ml | 42 |
| | 100 | μg/ml | 97 |
| Nimesulide | 0.3083 | ug/ml | 50 |

### Example 13

### 5-LO Inhibition

Human peripheral blood mononuclear leukocytes (PBML) were isolated by Ficoll-Paque density gradient centrifugation. Herbal preparation F or vehicle was preincubated with PBML (5×10⁶ cells/ml) in HBSS buffer, pH 7.4 at 37° C. for 15 minutes. After incubation with 30 μM A 23187 for 15 minutes, the reaction was terminated by addition of 1 N HCl. Following neutralization with NaOH and centrifugation, LTB₄ in the supernatant was measured using an EIA kit. 10 and 100 μg/ml Composition F were assayed.

As shown in Table 7, IC50 for preparation F was between 10 and 100 μg/ml. The IC50 for standard 5-LO inhibitor, Nordihydroguaretic acid, was 0.13 μM.

TABLE 7
Inhibition of 5-LO by preparation F

| | Concentration | | % Inhibition |
|---|---|---|---|
| Preparation F | 10 | μg/ml | 12 |
| | 100 | μg/ml | 90 |
| NDGA | 0.0393 | ug/ml | 50 |

NDGA = nordihydroguaretic acid

### Example 14

### MMP-3 Enzymatic Assay

MMP-3 (stromelysin-1, human recombinant catalytic domain from Calbiochem Cat. 444217) was activated by p-aminophenylmercuric acetate for 60 minutes at 37° C. After preincubation of preparation A or vehicle with the activated enzyme (5 nM) in a reaction mixture containing 50 mM MOPS (pH 7.2), 10 mM CaCl₂ and 10 μM ZnCl₂at 37° C. for 60 minutes, the reaction was initiated by addition of 4 μM (7-methoxycoumarin-4-yl)acetyl-Pro-Leu-Gly-Leu-N-3-(2,4-dinitrophenyl)-L-2,3-diaminopropinoyl-Ala-Arg-NH₂ and incubated for a further 120 minutes at 37° C. Enzyme activity was determined spectrofluorometrically by measuring the formation of fluorescent (7-methoxycoumarin-4-yl)acetyl-Pro-Leu-Gly.

As shown in Table 8, preparation A demonstrated a mild 12% inhibition of MMP-3 at 100 μg/ml.

TABLE 8
MMP-3 activity inhibition by preparation A

| Treatment | Concentration | | Inhibition (%) |
|---|---|---|---|
| TIMP-1 | 0.0076 | μM | 50 |
| TIMP-2* | 0.0077 | μM | 50 |
| Preparation A | 100 | μg/ml | 12 |

*Indicates standard reference agent used.

### Example 15

### MMP-8 Enzymatic Assay

MMP-8 (collagenase-2, stimulated human neutrophils, Calbiochem Cat. 444229) was activated by p-aminophenylmercuric acetate for 60 minutes at 37° C. After preincubation of preparation A or vehicle with the activated enzyme (6 nM) in a reaction mixture containing 50 mM MOPS (pH 7.2), 10 mM CaCl₂ and 10 μM ZnCl₂ at 37° C. for 60 minutes, the reaction was initiated by addition of 4 μM (7-methoxycoumarin-4-yl)acetyl-Pro-Leu-Gly-Leu-N-3-(2,4-dinitrophenyl)-L-2,3-diaminopropinoyl-Ala-Arg-NH₂ and incubated for a further 120 minutes at 37° C. Enzyme activity was determined spectrofluorometrically by measuring the formation of fluorescent (7-methoxycoumarin-4-yl) acetyl-Pro-Leu-Gly.

As shown in Table 9, preparation A demonstrated 18% inhibition of MMP-8 at 100 μg/ml.

TABLE 9
MMP-8 activity inhibition by preparation A

| Treatment | Concentration | | Inhibition (%) |
|---|---|---|---|
| TIMP-1 | 0.0039 | μM | 50 |
| TIMP-2* | 0.004 | μM | 50 |
| Preparation A | 100 | μg/ml | 18 |

*Indicates standard reference agent used.

### Example 16

### MMP-13 Enzymatic Assay

MMP-13 proenzyme (human recombinant, expressed in Sf9 cell, Calbiochem Cat. 444248) was activated by p-aminophenylmercuric acetate for 60 minutes at 37° C. After preincubation of preparation A or vehicle with the activated enzyme with the activated enzyme (0.5 nM) in a reaction mixture containing 50 mM MOPS (pH 7.2), 10 mM CaCl₂ and 10 μM ZnCl₂ for 60 minutes at 37° C., the reaction was initiated by addition of 4 μM (7-methoxycoumarin-4-yl)acetyl-Pro-Leu-Gly-Leu-N-3-(2,4-dinitrophenyl)-L-2,3-diaminopropinoyl-Ala-Arg-NH₂ and incubated for a further 120 minutes at 37° C. Enzyme activity was determined spectrofluorometrically by measuring the formation of fluorescent (7-methoxycoumarin-4-yl)acetyl-Pro-Leu-Gly.

As shown in Table 10, preparation A demonstrated 21% inhibition of MMP-13 at 100 μg/ml.

TABLE 10
MMP-13 activity inhibition by preparation A

| Treatment | Concentration | | Inhibition (%) |
|---|---|---|---|
| TIMP-1 | 0.0068 | μM | 50 |
| TIMP-2* | 0.0028 | μM | 50 |
| Preparation A | 100 | μg/ml | 21 |

*Indicates standard reference agent used.

### Example 17

### Free Radical Scavenger, SOD Mimetic Activity

Preparation A or vehicle was incubated with 0.12 mM xanthine, 6 mU xanthine oxidase, 27 μM nitroblue tetrazolium (NBT), 0.11 mM EDTA, 0.005% bovine serum albumin and Na₂CO₃ at pH 10.5 at 25° C. for 20 minutes. Conversion of xanthine to uric acid+O⁻+NBT to formazan was then determined by measurement of absorbance at 595 nm and percent inhibition by superoxide dismutase or test compound was calculated.

As shown in Table 11, preparation A demonstrated 50% SOD mimetic activity at 100 μg/ml.

TABLE 11
Free radical scavenger, SOD mimetic activity by preparation A

| Treatment | Concentration | | Inhibition (%) |
|---|---|---|---|
| (−)-Epicatechin gallate | 6.1 | μM | 50 |
| *Superoxide dismutase (SOD) | 0.0084 | μM | 50 |
| Preparation A | 100 | μg/ml | 50 |

*Indicates standard reference agent used.

### Example 18

### Human Use experiences

The assessment questionnaire was designed according to pain and movement of arthritis. 16 females with apparent arthritis symptoms were instructed to fill in the questionnaire at baseline and 12 weeks after intake of the composition of the invention extract powder at 900 mg/day, BID, by scoring the degree of pain and movement as None: 0, Slight: 1, Mild: 2, Moderate: 3, Severe: 4.

As shown in Table 12, there were significant decreases in pain, stiffness and difficulty in mobility. The decrease in lower back pain was most striking at 80%, while the pain in hip and knee decrease 60.2%. The difficulty in daily living mobility decreased 59.6%. The stiffness decreased 57.1%. All these parameters specific for assessment of arthritis decreased about 60% after three months of intake of the composition of the invention without apparent side effects.

TABLE 12
Change in pain and mobility of OA by the composition of the invention

| | Baseline | 12 Weeks | % Chang |
|---|---|---|---|
| Lower back pain | 26.0 | 8.5 | −67.3 |
| Hip or knee joint pain while: | | | |
| Walking on flat ground | 10.0 | 2.0 | −80.0 |
| Going up and down the stairs | 16.0 | 6.5 | −59.4 |
| Lying on bed | 10.5 | 4.5 | −57.1 |
| Sitting | 14.0 | 7.0 | −50.0 |
| Standing | 13.5 | 5.5 | −59.3 |
| Subtotal: | 64.0 | 25.5 | −60.2 |
| Stiffness: | | | |
| Wake up in the morning | 27.0 | 12.0 | −55.6 |
| After sitting, lying or resting | 25.5 | 10.5 | −58.8 |
| Subtotal: | 52.5 | 22.5 | −57.1 |
| Difficulty in mobility: | | | |
| Up and down stairs | 12.0 | 6.5 | −45.8 |
| Standing up and sitting down | 20.5 | 11.0 | −46.3 |
| Bending | 15.0 | 7.0 | −53.3 |
| Flat ground walking | 13.0 | 4.0 | −69.2 |
| In and out of car | 12.5 | 5.0 | −60.0 |
| Lying down and getting out of bed | 7.5 | 1.5 | −80.0 |
| Putting on and taking off socks | 14.0 | 4.0 | −71.4 |
| In and out of bath tub | 13.0 | 4.5 | −65.4 |
| Shopping | 3.0 | 2.5 | −16.7 |
| Heavy housekeeping work | 17.5 | 7.0 | −60.0 |
| Light housekeeping work | 10.5 | 3.0 | −71.4 |
| Subtotal: | 138.5 | 56.0 | −59.6 |
| Total | 281.0 | 112.5 | −60.0 |

While the invention has been described and exemplified in sufficient detail for those skilled in this art to produce and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. Other embodiments are set forth within the following claims.

What is claimed:

1. A composition for preventing or treating diseases or disorders caused by degeneration, and/or inflammation comprising (a) Lactuca sativa L.,(b) Aquilaria agallocha Roxb., and (c) Atractylodes macrocephala Koidz.

2. The composition of claim 1 wherein the diseases or disorders caused by degeneration and/or inflammation are arthritis and/or symptoms associated with arthritis.

3. The composition of claim 1 wherein the components (a), (b) and (c) are presented in dried, weight-to-weight ratios of about 5-20:2-10:1-6.

4. The composition of claim 3 wherein the weight-to-weight ratio is 10:5:3.

5. The composition of claim 4 which has the relative retention times of 6 major peaks relative to phenylbenzimidazole sulfonic acid in high performance liquid chromatography fingerprint being 0.70, 0.78, 1.24, 1.68, 1.87 and 2.12, respectively.

6. The composition of claim 5, wherein the peak at relative retention time 1.68 is chlorogenic acid.

7. The composition of claim 1 which repairs or regenerates articular joint surfaces.

8. The composition of claim 1 which has chondroprotective activities.

9. The composition of claim 1 which stimulates the proliferation of chondrocytes.

10. The composition of claim 1 which stimulates the synthesis of collagen and/or glycosaminoglycans in chondrocytes or bone marrow mesenchymal stem cells.

11. The composition of claim 1 which inhibits the production of nitric oxide in chondrocytes under fluid induced shear stress.

12. The composition of claim 1 which inhibits active or latent form of matrix metalloproteinase 2 produced by chondrocytes under fluid induced shear stress.

13. The composition of claim 1 which stimulates the production of tissue inhibitor of matrix metalloproteinase produced by chondrocytes under fluid induced shear stress.

14. The composition of claim 1 which stimulates expressions of genes selected from the group consisting of type II collagen, aggrecan, aggrecanase and bone morphogenetic protein-2 in chondrocytes under fluid induced shear stress.

15. The composition of claim 1 which inhibits matrix metalloproteinase 3.

16. The composition of claim 1 which inhibits matrix metalloproteinase 8.

17. The composition of claim 1 which inhibits matrix metalloproteinase 13.

18. The composition of claim 1 which has superoxide dismutase mimetic activities.

19. The composition of claim 1 which inhibits cyclooxygenase II.

20. The composition of claim 1 which inhibits lipooxgenase 5.

21. The composition of claim 1 further comprising pharmaceutically or food acceptable excipients, carriers or diluents.

22. The composition of claim 1 which is in the dosage form of lozenge, tablet, film coated tablet, capsule, soft capsule, granule, powder, pill, solution, emulsion, injection solution, injection, ointment, cream, spray, inhalant, soft gel, liquid, honey ball, or lotion.

23. A method of preventing or treating diseases or disorders caused by degeneration and/or inflammation comprising administering to a subject in need thereof an effective amount of extract of (a) Lactuca sativa L., (b) Aquilaria agallocha Roxb., and (c) Atractylodes macrocephala Koidz.

24. The method of claim 23, wherein the extract is administered by oral, subcutaneous injection, intra-muscular injection, intra-venous injection, intra-articular injection, mucosal membrane, transdermal or topical routes.

25. The method of claim 23, wherein the extract is administered in combination with a second therapeutic agent, and wherein the second therapeutic agent is selected from the group consisting of glucosamine, glucosamine sulfate, chondroitin sulfate, prednisone, dexamethasone, beclosmethasone, methylprednisone, betamethasone, hydrocortisone, methotrexate, cyclosporin, rapamycin, tacrolimus, antihistamine drugs, TNF antibodies, IL-1 antibodies, soluble TNF receptors, soluble IL-1 receptors, TNF or IL-I receptor antagonists, non-steroidal anti-inflammatory agents, COX-2 inhibitors.

26. The method of claim 23 wherein the diseases or disorders caused by degeneration and/or inflammation is arthritis or symptoms associated with arthritis.

27. The method of claim 23 wherein the components (a), (b) and (c) are presented in dried, weight-to-weight ratios of about 5-20:2-10:1-6.

28. The method of claim 27 wherein the weight-to-weight ratio is 10:5:3.

29. The method of claim 23 wherein the extract is effective to
(a) repair or regenerate articular joint surfaces;
(b) have chondroprotective activities;
(c) stimulate the proliferation of chondrocytes;
(d) synthesize collagen and/or glycosaminoglycans in chondrocytes or bone marrow mesenchymal stem cells;
(e) inhibit nitric oxide production in chondrocytes under fluid induced shear stress;
(f) inhibit active or latent form of matrix metalloproteinase 2 produced by chondrocytes under fluid induced shear stress;
(g) stimulate the expression of genes selected from the group consisting of type II collagen, aggrecan, aggrecanase and bone morphogenetic protein-2 in chondrocytes under fluid induced shear stress;
(h) inhibit matrix metalloproteinase-3, 8, 13;
(i) stimulate tissue inhibitor of matrix metalloproteinase produced by chondrocytes under fluid induced shear stress;
(j) have superoxide dismutase mimetic activity;
(k) inhibit cyclooxygenase II;
(l) inhibit lipooxgenase 5; or
a combination thereof.
